(19) United States
(12) Patent Application Publication
(10) Pub. No.: US 2022/0127157 A1
(43) Pub. Date: Apr. 28, 2022

(54) TITANATE CRYSTALS AND SURFACE TREATED TITANATE CRYSTALS

(71) Applicants: Miyoshi America, Inc., Dayville, CT (US); Otsuka Chemical Co., Ltd., Osaka-shi (JP); Miyoshi Kasei, Inc., Tokyo (JP)

(72) Inventors: Shoji TAKEKAWA, Dayville, CT (US); Ryo OHARA, Tokyo (JP); Nobuki ITOI, Tokushima (JP); Yasuhito ITO, Tokushima (JP); Hiroyoshi MORI, Tokushima (JP)

(21) Appl. No.: 17/078,731

(22) Filed: Oct. 23, 2020

(51) Int. Cl. C01G 23/00; A61K 8/29

(52) U.S. Cl. CPC C01G 23/005; A61K 8/29; C01G 23/006; C01P 2002/82; A61K 2800/412; A61K 2800/622; A61K 2800/614; C01P 2004/61

(57) ABSTRACT

The embodiments relate to cosmetic powder materials and compositions containing them that provide a high coverage to the skin and high near infrared/nitrated (NIR/IR) reflectance. The compositions include optionally surface treated titanate crystals used as cosmetic powders, and a cosmetically acceptable carrier. The compositions may be useful as a foundation and/or as a composition to correct skin discoloration, and may be used protect the skin from sun exposure.

### FIELD OF THE INVENTION

The embodiments relate to powder materials and compositions containing them. The embodiments provide a high coverage to the skin and high near infrared/infrared (NIR/IR) reflectance, in which the compositions include titanate crystals used as cosmetic powders, and a cosmetically acceptable carrier. The compositions may be useful as a foundation and/or as a composition to correct skin discoloration, and may be used to protect the skin from some sun exposure (NIR/IR). The powder materials also may be used in any number of applications in which powders may be used, and are not limited to use in cosmetic applications.

### BACKGROUND

The information provided below is not admitted to be prior art to the present invention, but is provided solely to assist in a more complete understanding of the embodiments.

A significant amount of various powders are conventionally used for making makeup, skincare products, toiletries, and other products marketed and distributed by the personal care industry. Powders dispersed in various product forms such as water base solution, water gel, w/o and o/w formulas, may suffer from poor dispersibility and product stability, which can result in the formation of aggregates, agglomerates and flocculation. These results can be due to the nature of powder's physical properties, including particle size, surface activity, charge, polarity and specific gravity, to name a few.

Untreated powder agglomerates easily, due to several surface properties (including surface charge, surface polarity etc.). In order to solve this problem and to thereby improve dispersibility and stability of powders, surface treatments with various treating agents have been proposed. Agents and methods for surface treating powders vary depending on the aim of the treatment. A treating agent may be selected in view of properties of the surface to be treated and its interaction with a dispersion medium. Known methods include, for instance, lipophilization with oils or metal soaps, hydrophilization treatment with surfactants or silica, and hydrophobization with silicone oils.

In recent years, powders have been developed to provide long lasting cosmetics with a smoother consistency. In obtaining these desirable traits, the focus has largely been on the hydrophobic properties of the surface treatments on powders and pigments, and improvements in the dispersibility of surface treated powders into an oil phase. However, when powders are used in cosmetic systems, such as foundations, lip sticks, lotions, sunscreens, or creams, the powders typically have to be dispersed in an aqueous phase, due to the hydrophilic nature of most cosmetic powders. To disperse non-hydrophobic powders in an aqueous phase, multiple emulsifiers are often used. Without these emulsifiers, dispersions in water-based systems often become problematic. The use of emulsifiers can be disadvantageous, however, with respect to producing a sticky, heavy feeling to the composition.

The affinity of a powder is dependent on surface characteristics of the powder, such as particle size, particularly nano-sized and micro-sized powders and the aspect ratio of powder. The high affinity of residual powders on the substrates that are commonly used in skin and hair consumer products often requires additional wash steps or specialized cleansing products to remove them completely. Furthermore, personal care products containing powders such as color pigments and various inorganic substrates (talc, mica, etc.), often make bathroom surfaces dirty, thus requiring cleaning.

It also is known that direct contact of inorganic and organic cosmetic powders with the skin may lead to the absorption of water on the skin surface, thus altering the natural hydrophilic and lipophilic balance, which may cause localized dehydration effects and consequently an unpleasant feeling by those using these products. In addition, the lack of homogeneity of the powders used, having different physical features from one another, may ultimately generate clearly perceptible defects. Cosmetic powders therefore are typically treated to modify the surface of the powder to provide improved dispersibility, homogeneity and stability and to reduce the deleterious effects caused by direct contact with the skin.

There are a variety of surface-treating methods generally available. In one method, a silicone oil (for instance, methyl polysiloxane, methyl hydrogen polysiloxane or alkyl silane with the number of carbon atoms of an alkyl portion being not more than 10) is dissolved into a solvent as a surface-treating agent, which then is added and mixed into a powder, and the surface treatment is baked onto the powder by heating after the drying process. In another method, while a powder and octyl triethoxy silane or the like are being dispersed into an organic solvent by using a media grinder, the surface of the powder is treated with an organic silicon compound such as octyl triethoxy silane (JP-A 08-104606). Another method involves stirring and mixing with a Henschel mixer N-octyl trimethoxy silane or N-octyl triethoxy silane as an alkyl silane compound, and a reaction is completed with the powder under heating, and the resultant treated powder is pulverized by a hammer mill (JP-A 2001-181136). In another method, a silicone compound such as methyl hydrogen polysiloxane or the like is emulsified by dispersing it in water, and surfaces of powder particles are coated by mixing the emulsion to the powder (JP-A 09-268271).

JP-B 06-59397 discloses a jet method in which after a metal soap, an organic silicon compound in which a reactive group such as a hydrogen group or the like is bonded to a silicon atom, and a powder are mixed, the mixture is pulverized by a miller using an ejecting stream simultaneously with the surface treatment. JP-A 2002-80748 discloses a method in which in order to improve dispersability of a powder, coating is effected with surface treating agents for an A layer and a layer B by a jet method. Another method involves mixing a silica compound in water, ethanol and aqueous ammonia, and therein dispersing titania powder to prepare a pre-mix 1. Separately, tetraethoxysilane, water and ethanol were mixed to prepare pre-mix 2. Pre-mix 2 was added to pre-mix 1 under stirring with a magnetic stirrer, at a constant rate over 2 hours. The mixture obtained was aged for 12 hours. The coating formation and aging were performed at 25° C. Thereafter, the solution was filtered by suction and the filtrate was dried with hot air at 50° C. for 12 hours to obtain silica-coated powder. This process is disclosed in U.S. Pat. No. 6,534,044, the disclosure of which is incorporated by reference herein in its entirety.

U.S. Pat. No. 5,496,544, the disclosure of which is incorporated by reference herein in its entirety, discloses a skin cosmetic composition consisting of an anhydrous powder comprising a solid powder phase mixed with a fat-based binder which contains a silicone mixture comprising at least one silicone oil, at least one silicone wax, at least one silicone resin, and optionally at least on silicone rubber and optionally at least one phenyl dimethicone. However, in U.S. Pat. No. 5,496,544, the anhydrous powder undergoes a physical treatment by the fat-based binder. Therefore, in the cosmetic composition from U.S. Pat. No. 5,496,544, the absence of a covalent chemical bond between the powder phase and fat-based binder has the drawback of an easy extraction of the latter from the powder phase. Also, in the cosmetic composition from U.S. Pat. No. 5,496,544, the powder phase coating consists of complex mixtures of silicones which confer a different kind of sensorial effects on the skin itself.

EP 1 116 753 describes a powder treated with reactive silicone comprising a powder surface-coated with a silicone compound, in which the amount of hydrogen generated from Si—H groups left on the surface of the silicone-treated powder is not greater than 0.2 ml/g of the treated powder and a contact angle between the water and the treated powder is at least 100°. However, the direct reaction between methyl hydrogen polysiloxane containing reactive Si—H bonds and the powder surface described in EP 1 116 753 fails to reach completion and it has the disadvantage to release some H₂ over time, which is the cause of several drawbacks for the obtained cosmetic powder. Indeed, on the one hand the generation of H₂ may cause the containers carrying the powder to swell and deteriorate, on the other hand the powder itself may harden and break.

Titanate crystals, such as lepidocrocite potassium magnesium titanates, have been described as useful friction materials used in brake pads and the like. For example, U.S. Pat. Nos. 7,078,009 and 7,307,047, the disclosures of which are incorporated by reference herein in their entireties, disclose lepidocrocite crystal powders having the formula K_{0.5-0.7}Li_{0.27}Ti_{1.73}O_{3.85-3.95}, and K_{0.2-0.7}Mg_{0.4}Ti_{1.6}O_{3.7-4} useful as friction control materials in brake pads, clutch facings, disc pads, and the like. Japanese Unexamined Patent Application Publication No. 2008-162971 discloses platy titanate crystal particles (K₃ₓLiₓTi₂₋ₓO₄, K₂ₓMgₓTi₂₋ₓO₄, or KₓFeₓTi₂₋ₓO₄, with 0.05≤x≤0.5 in every case) of the lepidocrocite type with an average particle size of 10˜100 μm, which can be used as bright pigment and bulking agent, and which provides a sunscreen effect. This publication discloses that lepidocrocite crystal powders having particle sizes outside the disclosed range may cause skin irritation, reduced ultraviolet shielding, reduced dispersibility, and loss of glossiness and transparency.

JP 4680757 and JP 4687588 disclose surface treating titanate powders with nonionic surfactants that adsorb to the surface of the powder, but are not chemically bound thereto, as well as lyophilizing the powder. The treated powders are said to have improved dispersibility.

What is needed in the art is a cosmetic powder that exhibits good dispersibility, good skin, nail, and hair coverage, and has improved protection from harmful radiation from exposure to the sun.

### BRIEF SUMMARY

According to a first aspect, an embodiment relates to an optionally surface treated titanate crystal particle useful as a powder for a variety of compositions and applications. In an embodiment, the titanate crystal particle has an average particle size of less than about 10 μm, and optionally has a surface treatment agent chemically bonded to its surface. In one embodiment the titanate crystal particle is selected from K_{0.5-0.8}Li_{0.27}Ti_{1.73}O_{3.85-4}, K_{0.2-0.8}Mg_{0.4}Ti_{1.6}O_{3.7-4}, and K_{0.5-0.8}Fe_{0.8}Ti_{1.6}O_{3.85-4} In another embodiment, the surface treatment agent chemically bonded to the surface of the titanate crystal particle is a hydrophobic surface treatment agent that renders the surface of the titanate crystal particle more hydrophobic (less hydrophilic) than it would be without the treatment. A variety of hydrophobic surface treatment agents useful in the embodiments include, for example, silicones, fatty acids, proteins, peptides, amino adds, N-acyl amino acids, monoglycerides, diglycerides, triglycerides, mineral oils, phospholipids, sterols, hydrocarbons, polyacrylates, and mixtures thereof.

According to another feature of an embodiment, there is provided a method of making a surface treated titanate crystal particle that includes: (a) preparing an aqueous solution of hydrophobic surface treatment agent; (b) adding to the aqueous solution at least one titanate crystal particle having an average particle size of less than about 5 μm with agitation to uniformly disperse the particle in the aqueous mixture; (c) adding a metal-containing salt to neutralize the aqueous mixture and chemically immobilize the hydrophobic surface treatment agent on the surface of the at least one titanate crystal particle; and (d) separating the surface treated titanate crystal particle from the aqueous mixture. The method also may include drying after separation.

According to another feature of an embodiment, there is provided a method of making a surface treated titanate crystal particle that includes: (a) preparing an aqueous solution of titanate crystal particle having an average particle size of less than about 10 μm; (b) adding to the aqueous solution at least one hydrophobic surface treatment agent with agitation to uniformly disperse the titanate crystal particle and the hydrophobic surface treatment agent in the aqueous mixture; (c) optionally adding a metal-containing salt to neutralize the aqueous mixture; (d) chemically immobilizing the hydrophobic surface treatment agent on the surface of the at least one titanate crystal particle; and (e) separating the surface treated titanate crystal particle from the aqueous mixture. The method also may include drying after separation.

According to other embodiments, there is provided a powder formulation that includes: (a) at least one optionally surface treated titanate crystal particle; and (b) a an acceptable carrier. According to other embodiments, there is provided a cosmetic formulation that includes a surface treated titanate crystal particle dispersion having a high concentration of surface treated titanate crystal particle.

The embodiments described herein provide powder compositions having improved coverage, uniformity, and duration of wear when applied to the skin. Still other aspects and advantages of the embodiments will become readily apparent to those having ordinary skill in the art from the following detailed description, wherein particularly preferred embodiments are shown and described, simply by way of illustration. As will be realized the preferred embodiments include other and different embodiments, and its several details are capable of modifications in various obvious respects. Accordingly, the description is to be regarded as illustrative in nature and not as restrictive.

### BRIEF DESCRIPTION OF THE DRAWINGS

FIG. 1 illustrates the infrared reflectance (IR) strength data from Example 5.

FIG. 2 illustrates the infrared reflectance (IR) strength data from Example 7.

### DETAILED DESCRIPTION OF THE INVENTION

The following definitions and non-limiting guidelines are provided to assist in better understanding the detailed description of herein. The headings (such as “Background” and “Brief Summary,”) and sub-headings used herein are intended only for general organization of topics within the disclosure of the embodiments, and are not intended to be limiting. For example, subject matter disclosed in the “Background” may include aspects of technology within the scope of the embodiments, and may not constitute a recitation of prior art. Subject matter disclosed in the “Brief Summary” is not an exhaustive or complete disclosure of the entire scope of the embodiments. Classification or discussion of a material within a section of the specification as having a particular utility (e.g., as being an “active” or a “carrier” ingredient) is made for convenience, and no inference should be drawn that the material must necessarily or solely function in accordance with its classification herein when it is used in any given composition.

The citation of references herein does not constitute an admission that those references are prior art or have any relevance to the patentability of the embodiments disclosed herein. Any discussion of the content of references cited in the Background is intended merely to provide a general summary of assertions made by the authors of the references, and does not constitute an admission as to the accuracy of the content of such references.

The description and specific examples, while indicating embodiments, are intended for purposes of illustration only and are not intended to be limiting. Moreover, recitation of multiple embodiments having stated features is not intended to exclude other embodiments having additional features, or other embodiments incorporating different combinations the stated of features. Examples are provided for illustrative purposes of how to make and use the compositions and methods described herein, unless explicitly stated otherwise, are not intended to be a representation that given embodiments have, or have not, been made or tested.

As used herein, the words “preferred” and “preferably” refer to embodiments that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope thereof. In addition, the compositions and the methods may comprise, consist essentially of, or consist of the elements described therein.

As used throughout, ranges are used as a short-hand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Throughout this description, the use of the term “about” or “approximately” is intended to denote an approximation of the number, which includes the number modified by the term, and a reasonable deviation from that term, including standard measurement errors. Unless otherwise specified, all percentages and amounts expressed herein and elsewhere in the specification should be understood to refer to percentages by weight. The amounts provided are based on the active weight of the material. The recitation of a specific value herein is intended to denote that value, plus or minus a degree of variability to account for errors in measurements. For example, an amount of 10% or about 10% may include 9.5% or 10.5%, given the degree of error in measurement that will be appreciated and understood by those having ordinary skill in the art.

As used herein, the term “cosmetic composition” means a composition that is intended to be applied onto the consumer's skin, particularly onto the facial skin or onto the body skin area or onto hair and nails, so as to regulate the condition of the skin and/or to improve the appearance of the skin, hair, and nails. The term “powder” without a numeric modifier denotes any material having a particle size within the range of from about 0.01 micrometer to 100 micrometers used for cosmetics. The term “average primary particle size” of powder denotes the equivalent volume mean primary particle size of the elementary powder. The average primary particle size is measured on the powder, before being treated.

Throughout this description, the term “foundation” means a cosmetic composition that is intended to be applied onto the consumer's skin, particularly, onto the facial skin, body skin, hair or nails so as to provide coverage and/or to mask skin irregularities and/or skin, hair, and nail imperfections and/or skin and nail tonal variations. The term “chalkiness” means the white hue which is observed onto skin after applying onto skin, particularly darker skin. The term “pastiness” means the white hue that may be observed on the skin after applying onto skin, particularly lighter skin.

All percentages, ratios and proportions herein are by weight, unless otherwise specified. All such weights as they pertain to listed ingredients are based on the active level, unless otherwise specified.

One of the features described herein is an optionally surface treated titanate crystal particle useful as a powder-containing composition, preferably for cosmetic compositions in which the titanate crystal particle has an average particle size of less than about 10 μm, and has a surface treatment agent chemically bonded to its surface. The surface treatment agent chemically bonded to the surface of the titanate crystal particle can be a hydrophobic surface treatment agent that renders the surface of the titanate crystal particle more hydrophobic (less hydrophilic) than it would be without the treatment. The embodiments described herein include both untreated and surface treated titanate crystal particles, with surface treated titanate crystal particles being particularly preferred.

An additional feature described herein is a method of making a surface treated titanate crystal particle that includes: (a) preparing an aqueous solution of hydrophobic surface treatment agent; (b) adding to the aqueous solution at least one titanate crystal particle having an average particle size of less than about 5 μm with agitation to uniformly disperse the particle in the aqueous mixture; (c) optionally adding a metal-containing salt to neutralize the aqueous mixture and chemically immobilize the hydrophobic surface treatment agent on the surface of the at least one titanate crystal particle; and (d) separating the surface treated titanate crystal particle from the aqueous mixture. The method also may include drying after separation.

According to another feature of an embodiment, there is provided a method of making a surface treated titanate crystal particle that includes: (a) preparing an aqueous solution of titanate crystal particle having an average particle size of less than about 10 μm; (b) adding to the aqueous solution at least one hydrophobic surface treatment agent with agitation to uniformly disperse the titanate crystal particle and the hydrophobic surface treatment agent in the aqueous mixture; (c) optionally adding a metal-containing salt to neutralize the aqueous mixture; (d) chemically immobilizing the hydrophobic surface treatment agent on the surface of the at least one titanate crystal particle; and (e) separating the surface treated titanate crystal particle from the aqueous mixture. The method also may include drying after separation.

Another feature described herein concerns a cosmetic formulation that includes: (a) at least one surface treated titanate crystal particle; and (b) a cosmetically acceptable carrier. According to other embodiments, there is provided a cosmetic formulation that includes a surface treated titanate crystal particle dispersion having a high concentration of surface treated titanate crystal particle.

The titanate crystal particles can be prepared in accordance with known techniques in the art. For example, methods of making such particles are described in U.S. Pat. Nos. 7,078,009 and 7,307,047, the disclosures of which are incorporated by reference herein in their entireties. The particles can be prepared by mixing, in the appropriate molar quantities, sources of potassium, lithium, magnesium, titanium, and iron, and then the addition of a flux and subsequent heating at elevated temperature. The particles produced then may be milled, dispersed in water or other appropriate liquid, to form an aqueous slurry. An acid then may be added to the aqueous slurry, the solid separated from the slurry, and heated at elevated temperatures to produce the titanate crystal particles. These particles then may be comminuted to the appropriate size, if needed.

The potassium source can be selected from compounds that produce potassium oxide when exposed to heat, specific examples of which include potassium oxide, potassium carbonate, potassium hydroxide, potassium nitrate, and the like. Such potassium sources may be used alone or in any combination, and the potassium source may be used in combination with a small amount of one or more of oxides, carbonates, hydroxides and nitrates of other alkaline metals. Magnesium sources include magnesium hydroxide, magnesium carbonate, magnesium fluoride and the like. Such magnesium sources may be used alone or in any combination. Examples of lithium sources include lithium hydroxide, lithium carbonate, lithium fluoride and the like. Such lithium sources may be used alone or in any combination. Iron sources likewise include iron oxides, iron carbonates, iron hydroxides, iron nitrates, and the like. The titanium source can be selected from any source of titanium, including titanium oxide-containing compounds, specific examples of which include titanium oxide, rutile ore, titanium hydroxide wet cake, water-containing titania, and the like. Such titanium sources may be used alone or in combination.

When preparing lithium titanate crystal , the potassium, lithium, and titanium sources can be mixed in their appropriate molar ratios, each deviating by ±10% in a suitable mixing apparatus. A particularly preferred lithium titanate crystal has the following formula: K_{0.5-0.8}Li_{0.27}Ti_{1.73}O_{3.85-4}.

When preparing magnesium titanate crystal, the potassium, magnesium, and titanium sources can be mixed in their appropriate molar ratios, each deviating by ±10% in a suitable mixing apparatus. A particularly preferred magnesium titanate crystal has the following formula: K_{0.2-0.8}Mg_{0.4}Ti_{1.6}O_{3.7-4}.

When preparing iron titanate crystal, the potassium, iron, and titanium sources can be mixed in their appropriate molar ratios, each deviating by ±10% in a suitable mixing apparatus. A particularly preferred iron titanate crystal has the following formula: K_{0.5-0.8}Fe_{0.8}Ti_{1.6}O_{3.85-4}.

After mixing the respective components together, a suitable flux then can be added to the mixture. Examples of fluxes include potassium chloride, potassium fluoride, potassium molybdenate, potassium tangstenate, and the like. Among those fluxes, potassium chloride is particularly preferred. The flux may be added to the raw material in the molar ratio (raw material:flux) of about 3:0.5 to about 3:20, or from about 3:1 to about 3:15, or from about 3:3.5-3:10.

The raw materials and flux then are heated at an elevated temperature, or calcined, using any apparatus suitable for heating the mixture. The mixture may be heated at a temperature between about 800 to about 1,500° C., or from about 900 to about 1,200° C., or from about 1,000 to about 1,100° C. The heating may take place for a period of from about 30 minutes to about 2 days, or from about 1 to about 24 hours. The heating temperature may be raised or lowered at any rate, but generally is adjusted at a rate of about 3 to about 7° C./min. After heating, the product may be wet disintegrated. Specifically, it may be crushed and ground using a jaw crusher, a bin mill and the like, dispersed in water and stirred in the form of a 5-10 wt. % aqueous slurry with a concentration of from about 1 to about 30% by weight, or from about 2 to about 25% by weight.

An acid then may be added to the aqueous slurry. Any suitable acid may be used, including those selected from inorganic acids such as sulfuric acid, hydrochloric acid and nitric acid; organic acids such as acetic acid; and the like. Such acids may be used in combination, if desired. The acid may be added to the aqueous slurry in an amount sufficient to maintain the aqueous slurry at a pH of 6-8, or at a pH of 6.5-7.5. The pH measurement of the aqueous slurry generally can be performed after addition of the acid and following about 1 to about 5 hours of stirring. The acid is generally used in the form of an aqueous solution. The concentration of the aqueous acid solution is not particularly specified and may be suitably chosen from a wide range. It may be generally maintained in the approximate range of 1-80 weight %.

Upon reaching a suitable pH, the solids present in the slurry then can be separated using any separation technique known in the art, including for example, filtering, centrifugation, and the like. The separated solids, which contain the titanate crystal particles, then can be washed, dried, and then subjected to an additional heating or calcination process. may be heated at a temperature between about 300 to about 1,000° C., or from about 350 to about 900° C., or from about 400 to about 700° C. The heating may take place for a period of from about 30 minutes to about 1 day, or from about 1 to about 12 hours. After heating, the resulting titanate crystal particles then can be subjected to comminution, pulverized, or passed through a suitable sieve to provide titanate crystal particles having an average particle size of less than about 10 μm.

The so-produced titanate crystal particles then can be surface treated with a surface treatment agent such that the surface treatment agent is chemically immobilized on the surface of the particle. In an embodiment, the surface treatment agent is a hydrophobic surface treatment agent, and chemical immobilization results from a bond formed between the surface of the titanate crystal particle and the surface treatment agent, typically via a metal. Chemical linkage or immobilization of the hydrophobic surface treatment agent to the titanate crystal differs from adsorption in that surface treated material has a more uniformly chemically bound reaction product. Chemical linkage or immobilization tends to reduce movement and/or rearrangement of any material linked or attached onto the surface of the modified titanate crystal particle. For example, a hydrophobic surface treatment agent, that is linked or attached to the surface of a titanate crystal particle will have less mobility than a treatment agent that is attached or linked to the surface of a titanate crystal particle by virtue of adsorption.

In order to facilitate or enhance immobilization of surface-treatment agents to the titanate crystal particle, a reaction may be created by a water soluble compound having a lipophilic or hydrophilic moiety being absorbed onto the surface of the titanate crystal particle. This moiety may come from a separately added component, such as a metal-containing salt, or it may be present on the surface of the surface treating agent. As a non-limiting example, addition of a water-soluble salt of a polyvalent metal, such as magnesium, calcium, aluminum, titanium, zinc or a zirconium salt (e.g., zirconium sulfate or chloride), or an alkaline salt, such as a sodium, potassium, lithium, ammonium, or an amine salt, can produce a chemical linkage. These metals typically are present in the form of a salt, such as a sulfate salt (e.g., aluminum sulfate, and the like). With the addition of a water soluble salt of a polyvalent metal for example, a chemical bond may be produced between the surface treatment agent and the titanate crystal particle surface. The reaction provides a surface-treatment agent chemically immobilized onto the surface of the titanate crystal particle. In contrast, conventional coating of a substrate, pigment, or other particle with a surface-treatment agent involves absorbing the surface-treatment agent onto the surface of the substrate, pigment, or other particle.

Following surface modification, the surface treated titanate crystal particle can then be admixed or blended with another (e.g., second) powder material, such as a different pigment, or substrate or extender, titanate crystal particle, or another cosmetically acceptable ingredient such as an oil, emulsifier, binder, etc. The second material may or may not have been treated with a surface treatment agent. Alternatively, two or more materials (e.g., different colored pigments), can be combined or mixed together prior to contact with a surface treatment agent, such as in an aqueous slurry, and then subsequently contacted with a hydrophobic surface treatment agent in order to simultaneously produce two or more surface modified or treated materials.

The titanate crystal particle whose surface has been modified with at least one hydrophobic surface treatment agent or salts thereof usually will have an average treatment ratio sufficient to render the surface of the titanate crystal particle more hydrophobic, to improve the titanate crystal's dispersability in cosmetic compositions, and to improve its spreading ability on the skin that results in improved coverage of the cosmetic product. Amounts of a surface treatment agent to employ in water based slurry compositions and production methods of the invention will vary depending upon the cosmetic, makeup, personal care or other product to be produced, or method of manufacture. For example, a surface-treatment agent may be used in an amount of at least 0.1% by weight (wt %), based on the weight of the titanate crystal material. Surface-treatment agents are typically present in an amount ranging from about 0.1 to about 200% by weight; or, from about 1.0 to about 60% by weight; or, from about 1.0 to about 30% by weight; or from about 1.0 to about 20% by weight, or from about 1.0 to about 5% by weight. Relatively low amounts of surface-treatment agents can also be used, e.g., 0.5, 1.5, 2.0, 3.0%, and the like

Suitable hydrophobic surface treatment agents are those that render the surface of the titanate crystal particle more hydrophobic (less hydrophilic) that it would be without the treatment. A variety of hydrophobic surface treatment agents useful in the embodiments include, for example, silicones, fatty acids, proteins, peptides, amino acids, N-acyl amino acids, monoglycerides, diglycerides, triglycerides, mineral oils, phospholipids, sterols, hydrocarbons, polyacrylates, and mixtures thereof.

Exemplary surface treatment agents, with moieties representing hydrophobic characteristics, include structures and salts of Formulas I-VIII:

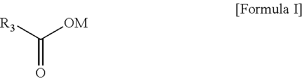

Wherein,
R₃ is an alkyl, alkylamide, alkenyl, alkynyl, alkoxy, aryl, cycloalkyl, arylalkyl group, all of which may be substituted by one or more hydroxy group, and may further be substituted by one or more alkoxyl, carboxyl, or oxo group;
R₁ is 8 to 24 carbons (C₈˜C₂₄); and
M is hydrogen, or metal or its equivalent (organic base such as triethanolamine, aminomethyl propanol, lysine, etc.).

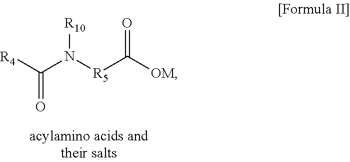

Wherein,
R₄ and R₅ are each independently alkyl, alkylamide, alkenyl, alkynyl, alkoxy, aryl, cycloalkyl, arylalkyl, amino acid group, all of which may be substituted by one or more hydroxy group, and may further be substituted by one or more alkoxyl, carboxyl, or oxo group;
R₄ is 8 to 24 carbons (C₈˜C₂₄);
R₁₀ is hydrogen or methyl; and
M is hydrogen, or metal or its equivalent (organic base such as triethanolamine, aminomethyl propanol, lysine, etc.).

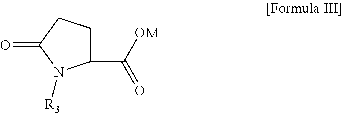

Wherein,
R₃ is an alkyl, alkylamide, alkenyl, alkynyl, alkoxy, aryl, cycloalkyl, arylalkyl group, all of which may be substituted by one or more hydroxy group, and may further be substituted by one or more alkoxyl, carboxyl, or oxo group;
Or R₃ is 8 to 24 carbons (C₈˜C₂₄); and
M is hydrogen, or metal or its equivalent (organic base such as triethanolamine, aminomethyl propanol, lysine, etc.).

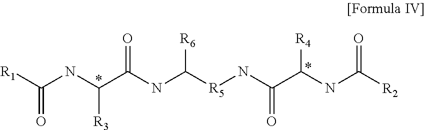

Wherein,
R₁ and R₂ is an alkyl, alkylamide, alkenyl, alkynyl, alkoxy, aryl, cycloalkyl, arylalkyl group, all of which may be substituted by one or more hydroxy group, and may further be substituted by one or more alkoxyl, carboxyl, or oxo group;
R₁ and R₂ are each independently 8 to 24 carbons (C₈˜C₂₄);
R₃ and R₄ are amino acid residual moieties;
R₅ and R₆ are an alkyl, alkylamide, alkenyl, alkynyl, alkoxy, aryl, cycloalkyl, arylalkyl group, all of which may be substituted by one or more hydroxy group, and may further be substituted by one or more alkoxyl, carboxyl, or oxo group; and
at least one of R₃, R₄ and R₆ has a carboxylic group, of which structure is either an acid form or a salt form, which is a metal, such as sodium, potassium, etc. or its equivalent; organic base such as triethanolamine, aminomethyl propanol, lysine, etc.

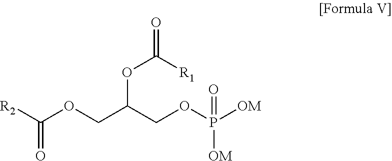

Wherein,
R₁ and R₂ are each independently alkyl, alkylamide, alkenyl, alkynyl, alkoxy, aryl, cycloalkyl, arylalkyl group, all of which may be substituted by one or more hydroxy group, and may further be substituted by one or more alkoxyl, carboxyl, or oxo group;
Or R₁ and R₂ are each independently 8 to 24 carbons (C₈˜C₂₄); and
M is hydrogen, or metal or its equivalent (organic base such as triethanolamine, aminomethyl propanol, lysine, etc.).

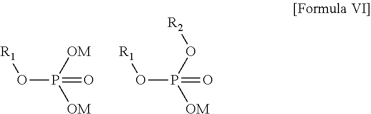

Wherein,
R₁ and R₂ are each independently alkyl, alkylamide, alkenyl, alkynyl, alkoxy, aryl, cycloalkyl, arylalkyl group, all of which may be substituted by one or more hydroxy group, and may further be substituted by one or more alkoxyl, carboxyl, or oxo group;
Or R₁ and R₂ are each independently 8 to 24 carbons (C₈˜C₂₄); and
M is hydrogen, or metal or its equivalent (organic base such as triethanolamine, aminomethyl propanol, lysine, etc.).

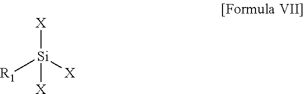

Wherein,
R₁ is an alkyl, alkylamide, alkenyl, alkynyl, alkoxy, aryl, cycloalkyl, arylalkyl group, all of which may be substituted by one or more hydroxy group, and may further be substituted by one or more alkoxyl, carboxyl, or oxo group;
Or R₁ is 8 to 24 carbons (C₈˜C₂₄); and
X is an alkoxy (e.g., methoxy, ethoxy, isopropoxy, isobutoxy, etc.) or a halogen (Cl, Br, etc.)

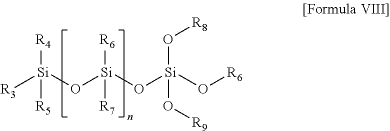

Wherein,
R₃, R₄, R₅, R₆, R₇, R₈, and R₉ are each independently an alkyl, alkyl amide, alkenyl, alkylnyl, alkoxy, aryl, cycloalkyl, or arylalkyl group, all of which may be substituted by one or more hydroxyl, alkyoxyl, carboxyl, or oxo groups; and
n is an integer from 1 to 60.

Substituent M in any of the above compounds can represent either a hydrogen or a metal or its equivalent. When M is a hydrogen, a carboxyl group forms and is thus present on the compound; when representing a metal or its equivalent, the salt of a carboxyl group forms and is thus present in the compound. Like any salt, a metal or equivalent retains an overall positive charge and the oxygen retains an overall negative charge. Exemplary metals include sodium, potassium, calcium, aluminum, and zinc; metal equivalents include amines such as monoethanolamine, diethanolamine, triethanolamine and ammonium, and organic bases such as lysine and arginine.

Alkyl, alkyl amide, alkenyl, alkylnyl, and alkoxy, groups as substituents set forth herein can be based upon alkyl groups having, for example, 1-24 carbon atoms. Such substituents can be fewer, for example, 1-20, 1-16, 1-12, 1-6 carbon atoms; such as aryl, cycloalkyl, and arylalkyl groups containing 6-24 carbon atoms, or fewer, e.g., 6-10 carbon atoms.

In particular embodiments in which there are two or more surface treatment agents, each of which are optionally chemically immobilized onto the surface of the titanate crystal particle, the first and the second surface treatment agent can be selected form any of the surface treatment agents set forth herein. Thus, for example, a first and a second surface treatment agent can be any of formulas I to VIII in any combination.

Examples of silicones useful as surface treatment materials include dimethicone, cyclomethicone, dimethiconol, dimethicone copolyol , dimethicone copolyol acetate, dimethicone copolyol butyl ether, dimethicone copolyol methyl ether, and mixtures thereof. Also useful are fluorinated, phenyl-substituted, and substituted derivatives of these silicones. Examples of fatty adds useful as surface treatment materials include C₁₀₋₅₀ straight or branched chain fatty adds, which can contain one or more unsaturated sites. Specific examples of fatty acids include capric acid, stearic acid, lauric acid, myristic acid, palmitic add, oleic acid, ricinoleic acid, linoleic acid, linolenic acid, eleostearic acid, arachidonic acid, and mixtures thereof. Also useful herein are metal salts (i.e. soaps) of the fatty acids. Useful metal salts of the fatty adds include sodium, potassium, magnesium, calcium, barium, aluminum, zinc, zirconium, titanium, and mixtures thereof. Examples of proteins useful as surface treatment materials Include collagen, chitin, casein, elastin, silk, and mixtures thereof. Examples of peptides include the partially hydrolyzed forms of the proteins just described wherein the proteins are broken down into a mixture of fragments each containing one or more amino acids.

Examples of amino acids useful as surface treatment materials include any of the naturally occurring amino acids, their N-methyl derivatives, and mixtures thereof. Also useful are salts of these materials. Preferred salts are those selected from the group consisting of aluminum, magnesium, calcium, zinc, zirconium, titanium, and mixtures thereof, with aluminum being most preferred. Examples of N-acyl amino acids useful as surface treatment materials include any of the amino acids, their N-methyl derivatives, or salts thereof just described, wherein the amino group has been acylated with a moiety derived from a C₁₀₋₅₀ straight or branched chain fatty acid. A particularly preferred N-acyl amino acid is disodium stearoyl glutamate.

Examples of mono-, di-, and triglycerides useful as surface treatment materials include those wherein the fatty acid portion of the molecule 1s derived from a C₁₀₋₅₀ straight or branched chain fatty add. Examples of mineral oils useful as surface treatment materials include mineral oil, petrolatum, and mixtures thereof. Examples of phospholipids useful as surface treatment materials include lecithin (which is also known as phosphatidyl choline), phosphatidyl ethanolamine, phosphatidyl—serine, phosphatidyl inoslitol, phosphatidyl acid, and mixtures thereof. An exemplary lecithin is hydrogenated lecithin. Examples of sterols useful as surface treatment materials include cholesterol and cholesterol esters wherein the acid portion of the ester is derived from a C1-30 straight or branched chain fatty acid,

Examples of hydrocarbons useful as surface treatment materials include polyethylene, polypropylene, polyisobutylene, squalane, squalene, and mixtures thereof. Also useful are fluorinated derivatives of polyethylene, polypropylene, and polyisobutylene. Examples of polyacrylates useful as surface treatment materials include polyacrylic add, polymethacrylic acid, polyethacrylic, and mixtures thereof.

Particularly preferred surface treatment agents include soaps (fatty acids/alkyl carboxylic acids salt), hydroxy fatty acids, alkyl sulfate, alkyl ether phosphate, polyoxyalkylene alkyl ether sulfate, polyoxyalkylene alkyl ether carboxylate, alkylether phosphate, acyl N-methyl taurate, N-acylamino acid salts (glutamate, sarcosinate, lalaninate, glycinate, B-alaninate), acyl peptides (acyl collagen, acyl silk protein), sodium cocoate, stearic acid, iso-stearic acid, potassium palmitate, sodium laurate, 12-hydroxystearic acid, sodium lauryl sulfate, sodium myristyl phosphate, sodium myristoyl sarcosinate, sodium polyoxyethylene lauryl sulfate, polyoxyethylene myristyl carboxylate, potassium myristate, zinc gluconate, isostearyl sebacic acid, sodium myristoyl taurate, disodium stearoyl glutamate, disodium cocoyl glutamate, arginine lauryl glycinate, sodium dilauramidoglutamide lysine. A particularly preferred hydrophobic surface treatment agent for use in the embodiments is a silicone.

Other suitable surface treatment agents may include one or more of the surface treatment agents disclosed in, for example, U.S. Pat. No. 6,887,494, U.S. Patent Application Publication Nos. 2008/0299158, 2011/0318286, the disclosures of which are incorporated by reference herein in their entireties.

The surface-modified titanate crystal particles can be prepared by preparing an aqueous solution by mixing at least water and titanate crystal particles to form a mixture. A binder such as an oil (emollient) may be added to the slurry (e.g., 1 to 180 parts of oil per 100 parts of titanate crystal particle) and dispersed. One or more surface treatment agents or a salt thereof, is dispersed into the slurry (e.g., about 0.5 to about 400 parts surface treatment agent per 100 parts titanate crystal particle) to the solution with high speed to homogenize the mixture to a homogenized powder mixture that is uniformly dispersed. The homogenized powder mixture then can be contacted with one to two chemical equivalents of a water-soluble salt of a polyvalent metal, such as an alkaline earth metal, calcium, magnesium, aluminum, titanium, zinc, or zirconium sulfate or chloride, to assist in linking the functional group of the surface-treatment agent to the surface of the titanate crystal particle, thereby chemically immobilizing the at least one surface treatment agent to the surface of the titanate crystal particle. Following surface treatment, the surface-modified titanate crystal particle is optionally dehydrated and rinsed to remove any secondary salts and byproducts, if necessary. A filtered cake is thereby produced which may optionally be further dehydrated to be “powder,” with less than about 10% loss on drying (LOD), for example, 5% LOD, or 3% LOD.

Other techniques useful in surface treating titanate crystal particles include dispersing the titanate crystal particles and surface treatment agent, (e.g., triethoxycaprylylsilane, etc.), and then heat the dispersion at an elevated temperature within the range of from about 75 to about 200° C., or above 100° C., to form a chemical bond between the surface treatment agent and the surface of the titanate crystal particle. Additional methods of surface treating pigments and other cosmetic powders that can be employed to prepare the surface-modified titanate crystal particles described herein are disclosed in, for example, U.S. Pat. Nos. 4,606,914; 4,622,074; and 5,368,639, the disclosures of which are incorporated by reference herein in their entireties. Specific methods disclosed therein are described briefly below.

Treatment with an N-acylamino treatment agent is disclosed in U.S. Pat. No. 4,606,914 as follows. First, into water is suspended a titanate crystal particle to form from about a 5 to 30% by weight suspension. To this suspension is added an N-acylamino acid water-soluble salt such as disodium stearoyl glutamate, in an amount of 0.5 to 10%, preferably 1 to 4%, by weight with respect to the titanate crystal particle and stirring is carried out to form a homogeneous suspension. By so treating, the pre-treatment of the titanate crystal particle, i.e., the primary particulation of the titanate crystal particle is promoted. While this suspension is being stirred, about 1 to 30%, preferably about 5 to about 10%, by weight of a water-soluble salt of Al, Mg, Ca, Zn, Zr and/or Ti as mentioned above is gradually added dropwise in such an amount that the water-soluble metal salt is about 0.65 to about 2 molar equivalents, preferably about 1 to about 1.2 molar equivalents, with respect to the N-acylamino acid water-soluble salt. Thereby, the N-acylamino acid water-soluble salt reacts with the water-soluble metal salt of Al, Mg, Ca, Zn, Zr and/or Ti to cause the N-acylamino acid metal salt to be successively orientated and adsorbed onto the surfaces of the titanate crystal particle. After the addition of the water soluble salt of Al, Mg, Ca, Zn, Zr and/or Ti, stirring is continued for about 10 min., followed by aging. Then, concentration is carried out by means of a centrifuge and drying is carried out at 80° to 120° C., thereby producing the surface-modified titanate crystal particle.

An example of a method for treating the surfaces of titanate crystal particles with hydrogenated lecithin, as disclosed in U.S. Pat. No. 4,622,074 follows. The titanate crystal particles to be treated are dispersed in water. Then the hydrogenated lecithin is added in a proportion of 0.3 to 10% by weight of hydrogenated lecithin, and the mixture is stirred vigorously with heating until it is completely dissolved or emulsified. At this point, a portion of the hydrogenated lecithin is adsorbed on the surface of the titanate crystal particles. In order to complete the adsorption of hydrogenated lecithin, a 1-30% by weight aqueous solution of water soluble salts of Al, Mg, Ca, Zn, Zr and Ti may be added dropwise, in sufficient amount to give a proportion of 0.1-2 equivalent of salt to hydrogenated lecithin. Examples of such metal salts include aluminum sulfate, aluminum chloride, aluminum nitrate, aluminum potassium sulfate, magnesium chloride, magnesium sulfate, magnesium nitrate, magnesium potassium sulfate, calcium chloride, calcium nitrate, calcium acetate, zinc chloride, zinc nitrate, zinc sulfate, zinc acetate, zirconium sulfate, zirconium chloride, titanium oxysulfate, and titanium chloride. The added metal salt reacts with hydrogenated lecithin to form a water insoluble reaction product which adsorbs onto the surfaces of the titanate crystal particles. In this embodiment, neutral fat (such as egg yolk oil and other oils) adsorbs simultaneously with the metal salt of hydrogenated lecithin onto the surfaces of the titanate crystal particles. The desired surface modified titanate crystal particles then can be obtained after removing water from the reaction mixture in a centrifuge, and drying at 80°-120° C. for a period of time ranging from about 5 to about 25 hours, or from about 10 to about 20 hours. The amount of hydrogenated lecithin adsorbed depends on the size of the titanate crystal particles. A thicker coating is usually obtained with particles of a smaller size.

An exemplary method for treating the surfaces of titanate crystal particles with dimethicone, as disclosed in U.S. Pat. No. 5,368,639 follows. According to this embodiment, the dimethicone-treated titanate crystal particle can be produced by mixing dimethicone, an organic solvent that dissolves dimethicone, and titanate crystal particles, and drying the mixture by heating. The dimethicone can be used in an amount of from about 0.1 to about 30%, or from 2-5 wt % by weight of the titanate crystal particles to be treated. The organic solvent (e.g., ethers, ketones, halogenated hydrocarbons, aliphatic hydrocarbons, alcohols, and mixtures thereof) can be used in an amount of from about 1-50 wt % by weight of the titanate crystal particles. Mixing may be accomplished by placing the components into a mixer, or by spraying dimethicone onto a mixture of titanate crystal particles and solvent. The mixture then can be heated to a suitable temperature to drive off the solvent, with consideration given to the heat resistance of the titanate crystal particles and the type of solvent used.

The so-prepared surface-modified titanate crystal particle then may be used in a powder-containing composition that contains conventional additives. For example, the so-prepared surface-modified titanate crystal particles may be used in cosmetic compositions that may or may not include conventional cosmetics additives, excipients, and the like. The composition may include additional coloring agents such as pigments and substrates. The composition also may contain up to about 25 wt % of a non-volatile oil. The non-volatile oil may be comprised of an organic, UV-active material that functions as a UV-protective agent (a “sun block”). Preferably, two or more organic, UV-actives are used to provide a wide spectrum of protection in the UV region. For example, a combination of at least one UV protecting agent that mainly provides protection against UVA light, and at least one UV protecting agent that mainly provides protection against UVB light, may be used.

The pigments useful in the embodiments include any known pigment including, for example, inorganic white pigments such as titanium dioxide and zinc oxide, inorganic red system pigments such as iron oxide (red iron oxide) and titanic acid irons, inorganic brown system pigments such as y-iron oxides, inorganic yellow system pigments such as yellow soil and yellow iron oxides, inorganic black color system pigments such as tetravalent acid iron oxide, carbon black, inorganic violet system pigments such as mango violet, cobalt violet, inorganic green system pigments such as chromium oxide, chromium hydroxide, and titanic acid cobalt, inorganic blue system pigments such as ultramarine blue, and prussian blue, pearl pigments such as titanium dioxide covered mica, titanium dioxide covered bismuth oxychloride, bismuth oxychloride, titanium dioxide covered talc, fish scale foil, colored titanium dioxide covered mica, metal powder pigment such as aluminum powder, copper powder, colored composite pigments such as iron-doped zinc oxide and iron-doped titanium dioxide.

Other pigments may be used, such as red No. 201, red No. 202, red No. 204, red No. 205, red No. 220, red No. 226, red No. 228, red No. 405, AP2199 Iron Oxide HP (commercially available from Elementis, Hightstown, N.J.), orange-colored No. 203, orange-colored No. 204, yellow No. 205, yellow No. 401 and blue No. 404, organic chlorophyll pigment such as FD&C Red No. 3, red No. 104, red No. 106, red No. 227, red No. 230, red No. 401, red No. 505, orange-colored No. 205, FD&C Yellow No. 4, yellow No. 5, yellow No. 202, yellow No. 203, orange-colored No. 3 and zirconium, barium, or aluminum lake of blue No. 1, natural colorants such as β-carotene, hydrocarbon oils such as squalane, mineral oil, vaseline, micro crystalline wax, ozokerite, ceresin, myristic acid, palmitic acid, stearic acid, oleic acid, iso-stearic acid, cetyl alcohol, hexadecyl alcohol, oleyl alcohol, cetyl 2-ethylhexanoate, 2-ethylhexyl palmitate, 2-octyldodecyl myristate, neo-pentylglycol di-2-ethylhexanoate, glyceryl tri-2-ethylhexanoate, 2-octyldocyl oleate, isopropyl myristate, glyceryl triisostearate, caprylic/capric triglyceride, olive oil, avocado oil, yellow bees wax, myristyl myristate, mink oil, lanolin oil, silicone oil, higher fatty acid oil, ester oils of fatty acids, higher alcohol, oil components of wax groups, cyclopentasiloxanes, dimethicones, trimethylsiloxysilicates, and organic solvents such as acetone, toluene, butyl acetate, and ester acetate can be used in various amounts.

Powder materials also may be utilized in accordance with the embodiments, and include any powder useful in cosmetic compositions including those that provide one or more of the following effects: luster effect, oil absorption, feeling improvement, cover effect, and the like. The particular powder used is not critical, and powders need not be used in various embodiments. Suitable powders include, for example, those belonging to the clay mineral group: such as those containing illite groups such as sericite (silky mica), muscovite, biotite, lithia mica, and synthetic mica; those containing kaolin groups such as kaolionite, nacrite, dekkite, halloysite; those containing sillimanite groups such as sillimanite and kyanite; magnesium silicate systems such as talc, and serpentine groups; and titanium dioxide and zinc dioxide.

The refractive index of oil solutions that are usually used in cosmetics typically is within the range of from about 1.39 to about 1.51, while the refractive index of mica is 1.59 and talc is 1.53, and the refractive index of the stratum corneum is 1.55. As can be seen from the numbers above, when normal pigments are used in cosmetics, the refractive indicies are very similar to each other, and when the pigments are wetted with skin-secreted sebum at its oil absorption or over this amount, some of the materials that make up the cosmetic film on the skin become transparent. When an excess amount of sebum is secreted, a reflection from the surface of the sebum film, as well as the reflection from the surface of the material dispersed within the sebum, will emphasize and create an undesirable shine, that when viewed from different angles will make the wrinkles stand out, and in occasion make the wearer of the cosmetic have a very tired look.

Depending on the formulation (e.g., liquid formulation, powder formulation, skin lotion, body soap, etc.), the amount of the surface modified titanate crystal particle can vary widely. For example, for a liquid formulation, the amount of the surface modified titanate crystal particle can be used in an amount of from about 0.5 to about 5% by weight, or from about 0.75 to about 3.5% by weight, or from about 1 to about 2.5% by weight, or at about 1% by weight. For a powder formulation, such as makeup foundation or the like, the surface modified titanate crystal particle can be used in an amount of from about 5 to about 65% by weight, or from about 10 to about 40% by weight, or from about 15 to about 35% by weight, or at about 20% by weight. For a skin lotion formulation, the surface modified titanate crystal particle can be used in an amount of from about 0 to about 30% by weight, depending on whether color is desired, or from about 0.5 to about 20% by weight, or from about 1 to about 10% by weight. For a body soap formulation, the surface modified titanate crystal particle can be used in an amount of from about 0 to about 30% by weight, depending on whether color is desired, or from about 0.5 to about 20% by weight, or from about 1 to about 10% by weight.

A wide variety of conventional UV protecting agents are suitable for use herein. Non-limiting exemplary organic, UV-actives include: 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene (Parsol 340, DSM), oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, avobenzone (Parsol 1789, DSM), avobenzone, PABA, octyldimethyl-PABA, Phenylbenzimidazole sulfonic acid, Cinoxate, Dioxybenzone (Benzophenone-8), Oxybenzone (Benzophenone-3), Homosalate, Menthyl anthranilate, Octisalate, Sulisobenzone, Trolamine salicylate, Terephthalylidene Dicamphor Sulfonic Acid, 4-Methylbenzylidene camphor, Methylene Bis-Benzotriazolyl Tetramethylbutylphenol, Bis-ethylhexyloxyphenol methoxyphenol triazine, bisimidazylate, Drometrizole Trisiloxane, Octyl triazone, Diethylamino Hydroxybenzoyl Hexyl Benzoate, Iscotrizinol, Polysilicone-15, Amiloxate, Ethylhexyl Dimethoxybenzylidene Dioxoimidazolidine Propionate, and mixtures thereof.

In addition to a UV-active, the non-volatile oil may comprise an ancillary oil which may be a solvent for one or more of the UV-active oils. The ancillary oil may provide desirable cosmetic properties such as emolliency and a good “skin feel.” A preferred, but non-limiting ancillary oil is isopropyl myristate. Non-volatile cosmetic emollient oils having a relatively high boiling point and function as a skin feel modifiers include, but are not hydrocarbons, fatty alcohols, fatty acids, non-volatile silicone oils, and esters such as glycerides and glycol esters.

Suitable ancillary oils include, but are not limited to isotridecyl isononanoate, isostearyl isostearate, isocetyl isosteatrate, isopropyl isostearate, isodecyl isonoanoate, cetyl octanoate, isononyl isononanoate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, egg yolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice germ oil, glycerol trioctanate, glycerol triisopalmiatate, trimethylolpropane triisostearate, glycerol tri-2-ethylhexanoate, pentaerythritol tetra-2-ethylhexanoate, lanolin, liquid lanolin, liquid paraffin, squalane, vaseline, and mixtures thereof. Commercially available oils include, for example, tridecyl isononanoate with tradename Crodamol TN available from Croda, Hexalan available from Nisshin Seiyu, and tocopherol acetates available from Eisai.

Non-volatile cosmetic emollients may include waxes such as, but not limited to paraffin wax, microcrystalline wax, ozokerite wax, ceresin wax, carnauba wax, candelilla wax, and eicosanyl behenate. Non-volatile silicon oils may be used including, but not limited to polymethylphenylsiloxane, polydiphenylsiloxane, polydiethylsiloxane, polydimethylsiloxane (dimethicone). For purposes of the present disclosure, a non-volatile silicon oil is defined as one that has a kinematic viscosity greater than 10 centiStokes (cSt). Suitable ancillary oils include polyalkyl or polyaryl siloxanes as disclosed in U.S. Pat. No. 6,936,241, the disclosure of which is incorporated by reference herein in its entirety.

Suitable ancillary oils useful herein include the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

The compositions useful in the embodiments described herein also may contain other conventional components useful in various compositions, preferably cosmetic compositions. Any cosmetically acceptable vehicle may be used together with the aluminum hydroxide coated with the pigment powder. Such vehicles may include, for example, water, glycerin, dimethicone, beeswax, glyceryl stearate, and the like. Other ingredients normally used in cosmetics also may be present, when desired. For example, inorganic powders such as talc, kaolin, sericite, muscovite, phlogopite, red mica, biotite, synthetic mica, lithia mica, vermiculite, magnesium carbonate, calcium carbonate, diatomite, magnesium silicate, calcium silicate, aluminum silicate, barium silicate, barium sulfate, strontium silicate, wolframic acid metal salt, or silica, hydroxyapatite, zeolite, boron nitride, ceramic powder, organic powders such as nylon powder, polyethylene powder, polystyrene powder, benzoguanamine powder, polyfluoridation ethylene powder, di-styrene benzene polymer powder, epoxy powder, acrylic powder, silicone powder, microcrystalline cellulose,

Resins such as alkyd resin, urea-formaldehyde resin, Nylon-12, plasticizers such as camphor, acetyl tributyl citric acid, ultraviolet absorbing agents, antioxidants, antiseptics, surfactants, moisturizing agents, perfumes, water, alcohol, and thickeners can also be used.

In accordance with one or more embodiments, compositions comprising the surface treated titanate crystal powders, as well as un-treated titanate crystal powders, preferably cosmetic compositions, have improved coverage as shown by a reduction in transmittance at 560 nm, when tested in accordance with the following testing protocol. The powder to be tested (about 10% by weight) is mixed together with an acrylate/dimethicone copolymer (about 90% by weight), and formed into a thin film on a suitable paper, such as for example self adhesive contact (TAC) paper. After the film has dried, the 560 nm transmittance is measured using a spectral haze meter, such as for example, an SH7000 haze meter commercially available from Nippon Denshoku Industries, Ltd., Japan. In one embodiment, the optionally surface treated titanate crystal containing compositions have a transmittance for light of the wavelengths from about 500 nm to about 650 nm of from about 65% to about 85%, or from about 70% to about 80%, or have a transmittance for light of the wavelength of about 560 nm of from about 75% to about 83%, it being understood that lower transmittance correlates with improved coverage. The titanate crystal particles disclosed herein preferably have a particle size of less than 10 μm, or a particle size less than a size selected from about 9, 8, 7, 6, 5, 4, 3, 2, or down to about 1 μm. It was found unexpectedly that decreasing the average particle size of the titanate crystal particle improved transmission, and that particle sizes of less than about 5 μm resulted in transmittance for light of the wavelength of about 560 nm of less than 80%. In an embodiment, the particle size of the titanate crystal particles range from about 1 to about 5 μm, or from about 2 to about 4.5 μm, or from about 2.5 to about 4.0 μm, or any value therebetween.

The surface treated powders described herein may be in any form and are not limited to particular one. For instance, the powders (or crystal particles) may be of powder, oil, water-in-oil emulsion, oil-in-water emulsion, nonaqueous emulsion, and multi-emulsion such as W/O/W or O/W/O. When used in a cosmetic composition, the composition may include skincare cosmetics such as a lotion, a milky lotion, a cream, a cleansing, a pack, an oil liquid, a massage agent, a beauty essence, a beauty oil, a detergent, a deodorant, a hand cream, a lip balm, and a wrinkle concealer; makeup cosmetics such as a make-up foundation, a concealer, a face powder, a powder foundation, a liquid foundation, a cream foundation, an oily foundation, a blush, an eye shadow, a mascara, an eyeliner, an eyebrow, and a lipstick; hair cosmetics such as a shampoo, a rinse, a treatments, and a setting agent; antiperspirants; and UV protective cosmetics such as a sunscreen oil, a sunscreen milky lotion, and a sunscreen cream. In addition, the surface treated powders described herein may be in various forms such as a liquid, emulsion, cream, solid, paste, gel, powder, pressed, laminated, mousse, spray, stick, and pencil forms.

In another embodiment, it was found that decreasing the amount of potassium in the titanate crystal particle improved coverage. For example, the amount of potassium can be decreased to about 50% of the original amount of potassium, or to about 25% of the original amount of potassium. For the lithium-containing titanate (K_{0.5-0.8}Li_{0.27}Ti_{1.73}O_{3.85-4}), a reduction in transmittance for light of the wavelength of about 560 nm of from about 6% to about 15%, or from about 8% to about 12%, or about 10.5% was achieved when the potassium content was reduced 50%, and a reduction of about 10% to about 25%, or from about 12% to about 18%, or about 15.2% was achieved when the potassium content was reduced to 25% of its original amount. For the magnesium-containing titanate (K_{0.2-0.8}Mg_{0.4}Ti_{1.6}O_{3.7-4}), a reduction in transmittance for light of the wavelength of about 560 nm of from about 3% to about 19%, or from about 4% to about 7%, or about 5.6% was achieved when the potassium content was reduced 50%, and a reduction of about 5% to about 15%, or from about 7% to about 10%, or about 8.1% was achieved when the potassium content was reduced to 25% of its original amount.

The following examples are intended to illustrate the invention. These examples should not be used to limit the scope of the invention, which is defined by the claims.

In the following examples, the FT-IR Measurement was carried out as follows: The Fourier transform infrared photometer FT-IR-5300 (Jasco Corporation) was used For the transmission spectrum, the ultraviolet-visible absorption spectral photometer (product name: UV-3150, Shimadzu Corporation) was used. The transmission spectrum was about 560 nm, and the sampling rate was 0.2 nm, and the measurement speed was slow speed.

### EXAMPLE 1

### Preparation of Lithium Potassium Titanate Surface Treated with Hydrogenated Lecithin

A lithium potassium titanate (Product A) was surface treated with hydrogenated lecithin (Egg Yolk Lecinol) using the materials listed in Table 1 below. 30.00 g of water (1) were mixed with 3.00 g of hydrogenated lecithin (Egg Yolk Lecinol), and the temperature was increase to a range of 70°-80° C. About 13.34 g of additional water (2) then were added and the temperature was adjusted to less than 40° C. A 10% aqueous solution of CaCl₂ (5.06 g) then was added to the mixture, and the surface treatment mixture was stirred for about 2 minutes. Product A (197 g) were introduced to a conventional mixer, such as a Kitchen Aid mixer, and then the surface treatment mixture was added and mixed for about 5 minutes. The mixer was scraped and mixed for an additional 5 minutes and the mixture heated to about 105° C. for about 16 hours to adequately dry the mixture. The dried particles then were pulverized in a 0.062 micron screen to produce the surface treated titanate crystals.

TABLE 1

| Material | Wt. % | Amount (g) |
|---|---|---|
| Product A * | 98.5 | 197.00 |
| Water (1) | 15.0 | 30.00 |
| Egg Yolk Lecinol | 1.50 | 3.00 |
| (hydrogenated Lecithin | | |
| Water (2) | 6.67 | 13.34 |
| CaCl₂ (10% aq.) | 2.53 | 5.06 |
| Total** | | 200 |

* Product A denotes lithium potassium titanate of the formula K_{0.5-0.8}Li_{0.27}Ti_{1.73}O_{3.85-4}
**Total amount after drying and evaporation of water.

### EXAMPLE 2

### Preparation of Lithium Potassium Titanate Surface Treated with Triethoxycaprylylsilane

A lithium potassium titanate (Product A) was surface treated with triethoxycaprylylsilane (Dynasylan Octeo-) using the materials listed in Table 2 below. Product A (197 g) were introduced to a conventional mixer, such as a Kitchen Aid mixer, and then 3.00 g of water was added and components were mixed for about 5 minutes. The mixer was scraped and mixed for an additional 5 minutes. To this mixture were added 4.00 g of triethoxycaprylylsilane (Dynasylan Octeo-) and the components were mixed for 5 minutes, the mixer then was scraped and the components were mixed for an additional 5 minutes. The mixture was heated to about 105° C. for about 16 hours to adequately dry the mixture. The dried particles then were pulverized in a 0.062 micron screen to produce the surface treated titanate crystals.

TABLE 2

| Material | Wt. % | Amount (g) |
|---|---|---|
| Product A * | 99.0 | 198.00 |
| Water (1) | 1.50 | 3.00 |
| Triethoxycaprylylsilane | 1.50 | 3.00 |
| Total** | | 200 |

* Product A denotes lithium potassium titanate of the formula K_{0.5-0.8}Li_{0.27}Ti_{1.73}O_{3.85-4}
**Total amount after drying and evaporation of water.

### EXAMPLE 3

### Preparation of Lithium Potassium Titanate Surface Treated with Disodium Stearoyl Glutamate

A lithium potassium titanate (Product A) was surface treated with disodium stearoyl glutamate (HS-21P) using the materials listed in Table 3 below. Product A (194 g) were introduced to a conventional mixer, such as a Kitchen Aid mixer. In a separate mixer, 6.0 g of HS-21P (disodium stearoyl glutamate) were dissolved in 110 g of hot deionized water (1), until fully dissolved, resulting in a clear mixture. This mixture then was added to the Product A, and mixed for 5 minutes. Additional water was added if needed. To this mixture then were added 9.12 g of water (2) and 2.28 g of a 10% aqueous solution of CaCl2 and mixed for about 5 minutes. The mixer was scraped and mixed for an additional 5 minutes and the mixture heated to about 105° C. for about 16 hours to adequately dry the mixture. The dried particles then were pulverized in a 0.062 micron screen to produce the surface treated titanate crystals.

TABLE 3

| Material | Wt. % | Amount (g) |
|---|---|---|
| Product A * | 97.0 | 194.00 |
| Water (1) | 55.0 | 110.0 |
| HS-21P (disodium stearoyl | 3.00 | 6.00 |
| glutamate) | | |
| Water (2) | 4.56 | 9.12 |
| CaCl₂ (10% aq.) | 1.14 | 2.28 |
| Total** | | 200 |

* Product A denotes lithium potassium titanate of the formula K_{0.5-0.8}Li_{0.27}Ti_{1.73}O_{3.85-4}.
**Total amount after drying and evaporation of water.

### EXAMPLE 4

### Preparation of Lithium Potassium Titanate Surface Treated with Dimethicone

A lithium potassium titanate (Product A) was surface treated with dimethicone (X24-9171) using the materials listed in Table 4 below. Product A (198 g) were introduced to a conventional mixer, such as a Kitchen Aid mixer, and then 40.0 g of water was added and components were mixed for about 5 minutes. The mixer was scraped and mixed for an additional 5 minutes. To this mixture were added 4.00 g of dimethicone (X24-9171) and the components were mixed for 5 minutes, the mixer then was scraped and the components were mixed for an additional 5 minutes. The mixture was heated to about 105° C. for about 16 hours to adequately dry the mixture. The dried particles then were pulverized in a 0.062 micron screen to produce the surface treated titanate crystals.

TABLE 4

| Material | Wt. % | Amount (g) |
|---|---|---|
| Product A * | 99.0 | 198.00 |
| Water (1) | 20.0 | 40.0 |
| X24-9171 (dimethicone) | 2.00 | 4.00 |
| Total | | 200 |

* Product A denotes lithium potassium titanate of the formula K_{0.5-0.8}Li_{0.27}Ti_{1.73}O_{3.85-4}.
**—Total amount after drying and evaporation of water.

### EXAMPLE 5

### Evaluation of IR Reflection

The surface treated titanate crystals of Example 4 (Product A) were compared to titanate crystals that were not surface treated (Product A′), and to MP-100 (a conventional TiO₂ particle having a large particle size) to assess the relative infrared reflection strength.

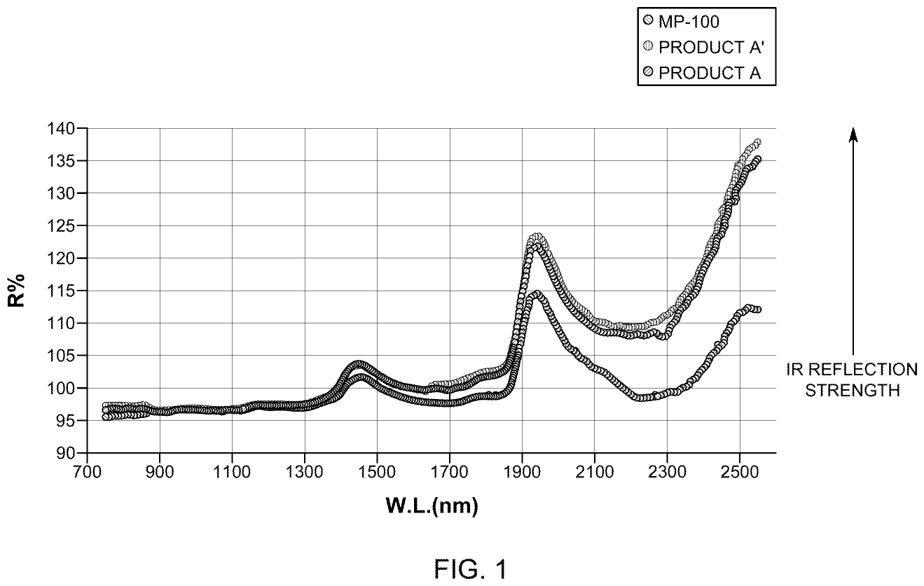

The results are shown in FIG. 1.

As shown in FIG. 1, the surface treated titanate crystals of Example 4 showed dramatically improved IR reflection strength, when compared to MP-100. The surface treated titanate crystals of Example 4 therefore had improved dispersibility when compared to un-treated powders, and consequently, the 560 nm coverage strength is improved.

### EXAMPLE 6

### Preparation of Magnesium Potassium Titanate Surface Treated with Triethoxycaprylylsilane

A magnesium potassium titanate (Product B) was surface treated with triethoxycaprylylsilane (Dynasylan Octeo-) using the materials listed in Table 5 below. Product B (198 g) were introduced to a conventional mixer, such as a Kitchen Aid mixer, and then 3.00 g of water was added and components were mixed for about 5 minutes. The mixer was scraped and mixed for an additional 5 minutes. To this mixture were added 4.00 g of triethoxycaprylylsilane (Dynasylan Octeo-) and the components were mixed for 5 minutes, the mixer then was scraped and the components were mixed for an additional 5 minutes. The mixture was heated to about 105° C. for about 16 hours to adequately dry the mixture. The dried particles then were pulverized in a 0.062 micron screen to produce the surface treated titanate crystals.

TABLE 5

| Material | Wt. % | Amount (g) |
|---|---|---|
| Product B * | 99.0 | 198.00 |
| Water (1) | 1.50 | 3.00 |
| Triethoxycaprylylsilane | 2.00 | 4.00 |
| Total** | | 200 |

* Product B denotes magnesium potassium titanate of the formula K_{0.2-0.8}Mg_{0.4}Ti_{1.6}O_{3.7-4}.
**Total amount after drying and evaporation of water.

### EXAMPLE 7

### Preparation of Magnesium Potassium Titanate Surface Treated with Dimethicone

A magnesium potassium titanate (Product B) was surface treated with dimethicone (X24-9171) using the materials listed in Table 6 below. Product B (198 g) were introduced to a conventional mixer, such as a Kitchen Aid mixer, and then 40.0 g of water were added and the components were mixed for about 5 minutes. The mixer was scraped and mixed for an additional 5 minutes. To this mixture were added 4.00 g of dimethicone (X24-9171) and the components were mixed for 5 minutes, the mixer then was scraped and the components were mixed for an additional 5 minutes. The mixture was heated to about 105° C. for about 16 hours to adequately dry the mixture. The dried particles then were pulverized in a 0.062 micron screen to produce the surface treated titanate crystals.

TABLE 6

| Material | Wt. % | Amount (g) |
|---|---|---|
| Product B * | 99.0 | 198.00 |
| Water (1) | 20.0 | 40.0 |
| X24-9171 (dimethicone) | 2.00 | 4.00 |
| Total | | 200 |

* Product B denotes magnesium potassium titanate of the formula K_{0.2-0.8}Mg_{0.4}Ti_{1.6}O_{3.7-4}
**—Total amount after drying and evaporation of water.

### EXAMPLE 8

### Evaluation of Coverage

This example compares the 560 nm transmittance results, which is indicative of the coverage ability of: (a) the cosmetic powder of Example 4, a surface treated lithium potassium titanate having the formula K_{0.5-0.8}Li_{0.27}Ti_{1.73}O_{3.85-4} that was surface treated with dimethicone in accordance with Example 4; (b) the cosmetic powder of Example 7, a surface treated magnesium potassium titanate having the formula K_{0.2-0.8}Mg_{0.4}Ti_{1.6}O_{3.7-4} that was surface treated with dimethicone in accordance with Example 7; and (c) other untreated powders. The 560 nm transmittance was measured in accordance with the methods described above. Table 7 provides the results of the experiments.

TABLE 7

| | 560 nm | 560 nm transmittance |
|---|---|---|
| Name | transmittance | Avg. |
| Surface treated TiO₂ | 77.11 | 78.57 |
| | 79.29 | |
| | 79.32 | |
| K_{0.2-0.8}Mg_{0.4}Ti_{1.6}O_{3.7-4} | 80.66 | 80.71 |
| (untreated) | 80.78 | |
| | 80.70 | |
| K_{0.2-0.8}Mg_{0.4}Ti_{1.6}O_{3.7-4} | 77.54 | 78.19 |
| (Silicone treated) | 78.08 | |
| | 78.94 | |
| Sericite | 91.39 | 90.79 |
| | 91.53 | |
| | 91.54 | |
| Talc | 90.96 | 90.96 |
| | 90.98 | |
| | 90.98 | |
| Kaolin | 89.05 | 89.04 |
| | 89.04 | |
| | 89.03 | |
| Mica | 89.67 | 89.71 |
| | 89.73 | |
| | 89.73 | |
| Boron Nitride | 89.71 | 89.85 |
| | 90.28 | |
| | 89.57 | |
| Silica | 87.74 | 87.49 |
| | 86.87 | |
| | 87.86 | |
| ZnO | 82.23 | 82.41 |
| | 82.47 | |
| | 82.53 | |

As shown in the above table, surface treated titanate crystals provided comparable and slightly improved coverage when compared to pigmentary grade TiO₂, the most popular and common cosmetic applied for purposes of coverage, and provided improved coverage when compared to Sericite, Talc, Kaolin, Mica, Boron Nitride, Silica, and ZnO, other popular and common cosmetic powders. ZnO typically is considered the 2^{nd} best cosmetic powder with respect to coverage, but the above table reveals that the surface treated titanate crystals provided significantly improved coverage with respect to ZnO.

### EXAMPLE 9

### Evaluation of IR Reflection

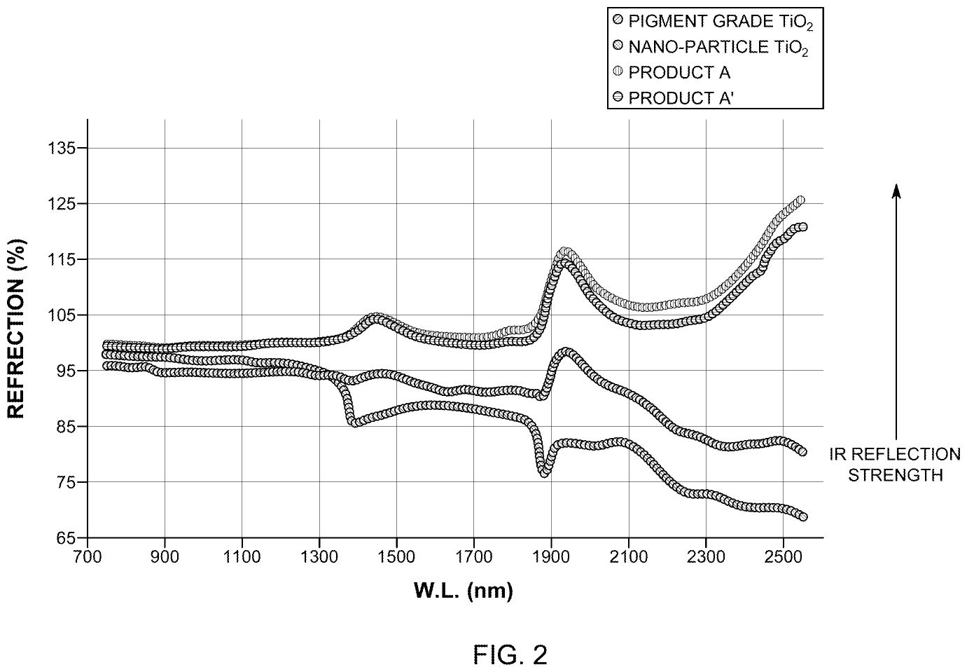

The surface treated titanate crystals of Example 4 (Product A), and non-surface treated lithium potassium titanate (Product A′), were compared to pigment grade TiO₂ to assess the relative infrared reflection strength. IR-strength was measured using a UV-3600Plus analyzer (commercially available from Shimadzu, Japan) having a 60 mm integrating sphere, the ISR-603, that is designed to measure absorbance and transmittance. The analyzer was set to a wavelength range of 2600-190 nm in the reflectance mode, slit width of 20, and 2.00 sampling interval. The results are shown in FIG. 2.

As shown in FIG. 2, the surface treated titanate crystals of Example 4 (Product A) and the non-surface treated titanate crystals (Product A′) showed dramatically improved IR reflection strength, when compared to TiO₂, which is commonly used for reflecting near infrared (NIR) and infrared (IR) rays. This property enables the use of the surface treated titanate crystals described herein for UV and IR/N-IR protection that is superior to the protection provided by one of the most popular protectant on the market today (TiO₂).

### EXAMPLE 10

### Evaluation of Particle Size on Coverage

The surface treated lithium potassium titanate crystals made in accordance with Example 4 above was subjected to grinding in increments of 15, 30, 45, and 60 minutes to reduce the particle size, and the various materials were subjected to the 560 nm transmittance test of Example 8 to evaluate coverage. The surface treated lithium potassium titanate crystals of various particle sizes were compared to a conventional white pigment, (triethoxycaprylylsilane surface treated TiO₂). The results are shown in Table 8 below.

TABLE 8

| | | 560 nm | Particle |
|---|---|---|---|
| | 560 nm | transmittance | Size D50 |
| Name | transmittance | Avg. | (μm) |
| surface treated TiO₂ | 43.16 | 43.35 | |
| | 43.31 | | |
| | 43.58 | | |
| Example 4 (as is) | 82.57 | 82.50 | 5.415 |
| | 82.55 | | |
| | 82.38 | | |
| Example 4 (15 min. | 78.39 | 78.56 | 4.341 |
| grind) | 78.25 | | |
| | 79.03 | | |
| Example 4 (30 min. | 77.25 | 77.31 | 3.992 |
| grind) | 76.75 | | |
| | 77.92 | | |
| Example 4 (45 min. | 76.97 | 77.11 | 3.752 |
| grind) | 76.88 | | |
| | 77.49 | | |
| Example 4 (60 min. | 75.5 | 75.53 | 3.054 |
| grind) | 75.53 | | |
| | 75.55 | | |

The data in the table above reveal that reducing the particle size of the surface treated titanate crystal particles of the embodiments described herein improves coverage. For example, it was observed that as the average particle size drops below 4 μm, the surface treated titanate crystal particles had comparable if not superior coverage when compared to surface treated TiO₂, a popular pigment used in cosmetic compositions.

### EXAMPLE 11

### Evaluation of Potassium Content on Coverage

The surface treated lithium potassium titanate crystals made in accordance with Example 4 above, and the surface treated magnesium potassium titanate crystals made in accordance with Example 7 above were evaluated as is, and with a reduced potassium content (50% of the original amount of potassium, and 25% of the original amount of potassium). The surface treated lithium potassium titanate crystals of various particle sizes were compared to a conventional white pigment, (triethoxycaprylylsilane surface treated TiO₂). The various materials were subjected to the 560 nm transmittance test of Example 8 to evaluate coverage, and the results are shown in Table 9 below:

TABLE 9

| | 560 nm | 560 nm transmittance |
|---|---|---|
| Name | transmittance | Avg. |
| surface treated TiO₂ | 43.16 | 43.35 |
| | 43.31 | |
| | 43.58 | |
| Example 4 (as is) | 82.57 | 82.50 |
| | 82.55 | |
| | 82.38 | |
| Example 4 (50% K) | 73.83 | 73.83 |
| | 73.82 | |
| | 73.84 | |
| Example 4 (25% K) | 69.98 | 69.98 |
| | 69.99 | |
| | 69.97 | |
| Example 7 (as is) | 86.14 | 86.06 |
| | 85.9 | |
| | 86.13 | |
| Example 7 (50% K) | 81.26 | 81.26 |
| | 81.26 | |
| | 81.26 | |
| Example 7 (25% K) | 79.09 | 79.12 |
| | 79.12 | |
| | 79.14 | |

The data in the table above reveal that reducing the potassium content of the surface treated titanate crystal particles of the embodiments described herein unexpectedly improves coverage. For example, it was observed that as the amount of potassium decreased, the surface treated titanate crystal particles had comparable if not superior coverage when compared to surface treated TiO₂, a popular pigment used in cosmetic compositions.

### EXAMPLE 12

### Evaluation of Particle Size and Reduced Potassium Amount on Coverage

The surface treated lithium potassium titanate crystals made in accordance with Example 4, with the potassium content reduced to 25% of its original amount as prepared in Example 11 above was subjected to grinding in increments of 15, 30, 45, and 60 minutes to reduce the particle size, and the various materials were subjected to the 560 nm transmittance test of Example 8 to evaluate coverage. The surface treated lithium potassium titanate crystals of various particle sizes were compared to a conventional white pigment, (triethoxycaprylylsilane surface treated TiO₂). The results are shown in Table 10 below.

TABLE 10

| | | 560 nm | Particle |
|---|---|---|---|
| | 560 nm | transmittance | Size D50 |
| Name | transmittance | Avg. | (μm) |
| surface treated TiO₂ | 43.16 | 43.35 | |
| | 43.31 | | |
| | 43.58 | | |
| Examples 4 and 11— | 69.98 | 69.98 | 4.21 |
| 25% K (as is) | 69.99 | | |
| | 69.97 | | |
| Examples 4 and 11— | 69.39 | 68.78 | 3.43 |
| 25% K (15 min. grind) | 68.66 | | |
| | 68.28 | | |
| Examples 4 and 11— | 67.06 | 66.32 | 2.45 |
| 25% K (30 min. grind) | 66.09 | | |
| | 65.81 | | |
| Examples 4 and 11— | 65.70 | 65.67 | 2.22 |
| 25% K (45 min. grind) | 64.85 | | |
| | 66.46 | | |
| Examples 4 and 11— | 62.65 | 63.07 | 2.54 |
| 25% K (60 min. grind) | 62.71 | | |
| | 63.84 | | |

The data in the table above reveal that reducing the particle size of the surface treated titanate crystal particles with a reduced potassium content greatly and unexpectedly improved coverage. For example, it was observed that a reduced potassium content surface treated titanate particle exhibited about a 9.7% improved coverage when compared to surface treated TiO₂, a popular pigment used in cosmetic compositions. It was further observed that the coverage was further improved as the particle size was reduced (15 min grind—11.3% improvement; 30 min grind—14.5% improvement; 45 min grind—15.3% improvement; and 60 min grind—18.7% improvement).

While the embodiments have been described with reference to specific examples and features, persons having ordinary skill in the art will appreciate that various modifications may be made to the embodiments without departing from the spirit and scope thereof.

What is claimed is:

1. A titanate crystal particle having an average particle size of less than about 10 μm, comprising a surface treatment agent chemically bonded to its surface, wherein the titanate crystal particle is selected from K_{0.5-0.8}Li_{0.27}Ti_{1.73}O_{3.85-4}, K_{0.2-0.8}Mg_{0.4}Ti_{1.6}O_{3.7-4}, and K_{0.5-0.8}Fe_{0.8}Ti_{1.6}O_{3.85-4}.

2. The titanate crystal particle of claim 1, wherein the titanate crystal particle is lithium potassium titanate having the formula K_{0.57-0.8}Li_{0.27}Ti_{1.73}O_{3.85-4}.

3. The titanate crystal particle of claim 1, wherein the titanate crystal particle is magnesium potassium titanate having the formula K_{0.53-0.8}Mg_{0.4}Ti_{1.6}O_{3.7-4}.

4. The titanate crystal particle of claim 1, wherein the titanate crystal particle is iron potassium titanate having the formula K_{0.53-0.8}Fe_{0.8}Ti_{1.6}O_{3.85-4}

5. A cosmetic containing the titanate crystal particle according to claim 1.

6. The titanate crystal particle of claim 1, wherein the surface treatment agent is selected from the group consisting of silicones, fatty acids, proteins, peptides, amino acids, N-acyl amino acids, monoglycerides, diglycerides, triglycerides, mineral oils, phospholipids, sterols, hydrocarbons, polyacrylates, and mixtures thereof.

7. The titanate crystal particle of claim 1, wherein the surface treatment agent is one or more silicones selected from the group consisting of dimethicone, cyclomethicone, dimethiconol, dimethicone copolyol, dimethicone copolyol acetate, dimethicone copolyol butyl ether, dimethicone copolyol methyl ether, and mixtures thereof.

8. The titanate crystal particle of claim 1, wherein the surface treatment agent is selected from the group consisting of dimethicone, disodium stearoyl glutamate, hydrogenated triethoxycaprylylsilane, and mixtures thereof.

9. The titanate crystal particle of claim 1, wherein the surface treatment agent is present in an amount within the range of from about 1 to about 20% by weight, by weight of the titanate particle.

10. The titanate crystal particle of claim 1, wherein the average D50 particle size is less than about 8 μm.

11. The titanate crystal particle of claim 1, wherein the amount of potassium is reduced by an amount within the range of from about 25% to 75% of the original amount of potassium in the titanate crystal particle.

12. A method of making titanate crystal particles coated with at least one surface treatment agent, comprising:
a) preparing slurry by mixing at least one titanate crystal particle selected from the group consisting of K_{0.5-0.8}Li_{0.27}Ti_{1.73}O_{3.85-4}, K_{0.2-0.8}Mg_{0.4}Ti_{1.6}O_{3.7-4}, and K_{0.5-0.8}Fe_{0.8}Ti_{1.6}O_{3.85-4} and at least one solvent fora period of time sufficient to adequately disperse the titanate crystal particles in the at least one solvent;
b) adding at least one surface treatment agent to the slurry, and mixing the components for a period of time sufficient to adequately mix the at least one surface treatment agent and the slurry to form a surface treated slurry;
c) heating the surface treated slurry at a temperature and for a time sufficient to remove the at least one solvent to form a dry mixture; and
d) pulverizing the dry mixture to form surface treated titanate crystal particles having an average particle size of less than about 10 μm.

13. The method as claimed in claim 12, wherein the surface treated slurry is heated to a temperature within the range of from about 80 to about 120° C. for a period of time of from about 10 to about 20 hours.

14. The method as claimed in claim 12, wherein the wherein the titanate crystal particle is lithium potassium titanate having the formula K_{0.57-0.8}Li_{0.27}Ti_{1.73}O_{3.85-4}.

15. The method as claimed in claim 12, wherein the wherein the titanate crystal particle is lithium potassium titanate having the formula K_{0.53-0.8}Mg_{0.4}Ti_{1.6}O_{3.7-4}.

16. The method as claimed in claim 12, wherein the surface treatment agent is selected from the group consisting of dimethicone, triethoxycaprylylsilane, and mixtures thereof.

17. A method of making titanate crystal particles coated with at least one surface treatment agent, comprising:
a) preparing a solution or slurry by mixing at least one surface treatment agent and at least one solvent at a temperature and for a period of time sufficient to adequately disperse or dissolve the titanate crystal particles in the at least one solvent to form a solution or slurry;
b) adding to the solution or slurry of a) at least one titanate crystal particle selected from the group consisting of K_{0.5-0.8}Li_{0.27}Ti_{1.73}O_{3.85-4}, K_{0.2-0.8}Mg_{0.4}Ti_{1.6}O_{3.7-4}, and K_{0.5-0.8}Fe_{0.8}Ti_{1.6}O_{3.85-4} and mixing for a period of time sufficient to form a titanate particle mixture;
c) adding a metal-containing salt to the titanate particle mixture, and mixing for a period of time sufficient to facilitate chemical immobilization of the surface treatment agent on the at least one titantate crystal particle and form a surface treated mixture;
d) heating the surface treated mixture at a temperature and for a time sufficient to remove the at least one solvent to form a dry mixture; and
e) pulverizing the dry mixture to form surface treated titanate crystal particles having an average particle size of less than about 10 μm.

18. The method as claimed in claim 17, wherein the surface treated mixture is heated to a temperature within the range of from about 80 to about 120° C. for a period of time of from about 10 to about 20 hours.

19. The method as claimed in claim 17, wherein the surface treatment agent is selected from the group consisting of disodium stearoyl glutamate, hydrogenated lecithin, and mixtures thereof.

20. A method of making 7 titanate crystal particles coated with at least one surface treatment agent, comprising:
a) preparing a solution or slurry by mixing at least one surface treatment agent and at least one solvent at a temperature and for a period of time sufficient to adequately disperse or dissolve the titanate crystal particles in the at least one solvent to form a solution or slurry;
b) adding a metal-containing salt to the solution or slurry, and mixing for a period of time sufficient to form a surface treatment mixture;
c) adding to the surface treatment mixture of b) at least one titanate crystal particle selected from the group consisting of K_{0.5-0.8}Li_{0.27}Ti_{1.73}O_{3.85-4}, K_{0.2-0.8}Mg_{0.4}Ti_{1.6}O_{3.7-4}, and K_{0.5-0.8}Fe_{0.8}Ti_{1.6}O_{3.85-4} and mixing for a period of time sufficient to term surface treated mixture;
d) heating the surface treated mixture at a temperature and for a time sufficient to remove the at least one solvent to form a dry mixture; and
e) pulverizing the dry mixture to form surface treated titanate crystal particles having an average particle size of less than about 10 μm.

21. The method as claimed in claim 20, wherein the surface treated mixture is heated to a temperature within the range of from about 80 to about 120° C. for a period of time of from about 10 to about 20 hours.

22. The titanate crystal particle of claim 1, wherein the average D50 particle size is within the range of from 2 to about 4.5 μm.

23. A titanate crystal particle having an average particle size of less than about 5 μm, wherein the titanate crystal particle is selected from K_{0.5-0.8}Li_{0.27}Ti_{1.73}O_{3.85-4}, K_{0.2-0.8}Mg_{0.4}Ti_{1.6}O_{3.7-4}, and K_{0.5-0.8}Fe_{0.8}Ti_{1.6}O_{3.85-4}

24. The titanate crystal particle of claim 23, wherein the titanate crystal particle is lithium potassium titanate having the formula K_{0.57-0.8}Li_{0.27}Ti_{1.73}O_{3.85-4}.

25. The titanate crystal particle of claim 23, wherein the titanate crystal particle is magnesium potassium titanate having the formula K_{0.53-0.8}Mg_{0.4}Ti_{1.6}O_{3.7-4}.

26. The titanate crystal particle of claim 23, wherein the average D50 particle size is within the range of from 2 to about 4.5 μm.

27. A cosmetic containing the titanate crystal particle according to claim 2.

28. A cosmetic containing the titanate crystal particle according to claim 3.
A cosmetic containing the titanate crystal particle according to claim 4.
